(19) United States
(12) Patent Application Publication
(10) Pub. No.: US 2025/0303025 A1
(43) Pub. Date: Oct. 2, 2025

(54) SCAFFOLD FOR SOFT TISSUE AUGMENTATION AND REINFORCEMENT

(71) Applicant: SMARTFEM MEDICAL TECHNOLOGY PTY LTD, KENT TOWN (AU)

(72) Inventor: Fariba BEHNIA-WILLISON, KENT TOWN (AU)

(21) Appl. No.: 18/688,041

(22) PCT Filed: Aug. 31, 2022
(86) PCT No.: PCT/AU2022/051064

§ 371 Date: Feb. 29, 2024

(30) Foreign Application Priority Data
Aug. 31, 2021 (AU) 2021902826
Mar. 3, 2022 (AU) 2022900504

(51) Int. Cl. A61L 27/36; A61F 2/00; A61L 27/22

(52) U.S. Cl. CPC A61L 27/3616; A61F 2/0045; A61L 27/225; A61L 27/3687; A61F 2220/0008; A61F 2240/001; A61L 2430/22

(57) ABSTRACT

The disclosure relates to a scaffold graft material, or platelet rich plasma (PRP) graft, and method of production thereof, for tissue repair and/or support (e.g. reinforcement). In one particular application, the scaffold material is used for treating pelvic organ prolapse (POP).

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### CROSS-REFERENCE TO RELATED APPLICATIONS

This application claims priority from AU2021902826 filed 31 Aug. 2021 and AU2022900504 filed 3 Mar. 2022, the entire contents of which are herein incorporated by reference.

### FIELD OF THE INVENTION

The disclosure relates to a scaffold graft material, and method of production thereof, for tissue repair and/or support (e.g., reinforcement). In one particular application, the scaffold material is used for treating pelvic organ prolapse (POP).

### BACKGROUND

Pelvic organ prolapse (POP), including vaginal prolapse is a common condition in women with an incidence of 40-60% and a lifetime risk of surgery of 12-19%. In Australia, for example, it has been estimated that over 50% of women who have given birth vaginally are at risk of POP to some degree (www.contienence.org.au). However, childbirth is not the only cause of POP in women (e.g., gynaecological cancer treatment and heavy lifting, age and obesity are also common causes; Ramaseshan A S et al., Int Urogynecol J 29(4):459-476, 2018), so that the actual incidence of POP among Australian women can be expected to be significantly higher.

POP is characterised by a descent of the pelvic organs (e.g., vagina, bladder, uterus and bowel) from their normal locations typically due to the “collapse” of the pelvic floor, particularly due to weakness and/or tears/stretching of the pelvic floor muscles and supporting tissues. In women, there are generally three recognised types of POP; namely, anterior vaginal wall prolapse (which can be characterised by a protuberance of the bladder and/or urethra into the front wall of the vagina), posterior vaginal wall prolapse (which is characterised by a portion of the bowel or rectum protruding into the back wall of the vagina) and apical vaginal prolapse wherein the uterus, or the top part of vagina, may drop down into the vaginal canal. In any case, POP can cause a range of different symptoms including vaginal bulge, discomfort (e.g., a feeling of pressure or fullness), difficult bowel movements, and loss of bladder control (e.g., difficultly in urinating or, commonly, urinary leakage or incontinence).

In many cases, POP in women can be managed effectively by regular exercise (e.g., Kegel exercises to strengthen the pelvic floor muscles) and lifestyle choices or changes (e.g., the risk of developing POP is significantly increased by smoking and overweight; www.voicesforpfd.org), and/or the use of a pessary or vaginal support device. However, in more serious cases, surgical intervention may be recommended, particularly for women with more severe symptoms related to quality of life, bladder, bowel and sexual function. Numerous reconstructive and obliterative surgical approaches are available for POP, and until relatively recently, the use of synthetic mesh (i.e. polypropylene transvaginal mesh) was commonly employed, particularly for surgery for a cystocele (bladder protruding into the front wall of the vagina).

However, due to complications such as mesh erosion, dyspareunia, hispareunia, pelvic pain and increased re-operation rates and associated reduced quality of life, the use of synthetic mesh for POP surgery has been withdrawn in Australia and other countries. Consequently, gynaecological surgeons are returning to native tissue repair (NTR), however such techniques show a high failure rate (e.g., in one study investigating NTR for stage 2 anterior vaginal wall prolapse, it was found that 33% of patients required secondary prolapse compartment procedures from 0.6 to 13 years later; Lavelle R S et al., J Urol 195(4 Pt 1):1014-1020, 2016).

To this end, trials have been conducted using various biological grafts (e.g., human dermal cadaveric allografts and mammalian extracellular matrix (ECM) xenografts) to augment POP surgery, but it has been considered that there is insufficient evidence to support their use over standard NTR techniques (Advances in Female Pelvic Medicine and reconstructive Surgery, eds. HW Brown and RG Rogers, Elsevier, Philadelphia, PA, United States of America, 2021), and at this time, in Australia at least, such biological grafts are unavailable for POP surgery due to the lack of evidence of efficacy, and also as an aftermath of the abovementioned synthetic mesh complications (www.tga.gov.au/alert/tga-actions-after-review-urogyaecological-surgical-mesh-implants#actions).

Other surgical techniques using biological grafts or absorbable mesh to augment POP have been trialled, with systemic reviews based on low quality evidence demonstrating minimal advantage compared with NTR regarding rates on awareness of prolapse or reoperation (Maher C, Feiner B, Baessler K, Christmann-Schmid C, Haya N, Marjoribanks J. Transvaginal mesh or grafts compared with native tissue repair for vaginal prolapse. Cochrane Database Syst Rev. 2016; 2:CD012079). Low to moderate quality evidence suggests higher recurrence rates for anterior prolapse after NTR that with biological grafts.

Thus, there is an urgent need for the identification and development of novel non-mesh approaches for the treatment of POP such as techniques which may support and enhance pelvic floor native tissue repair as well as reduce the failure rate following surgery.

### SUMMARY

The present disclosure is based on products derived from whole blood which facilitate augmentation of native tissue repair and to provide tissue augmentation and physiological support. Disclosed herein are compositions (scaffolds) and methods for producing such compositions (scaffolds) which are particularly useful for providing mechanical support in pelvic organ prolapse.

In a first aspect, the present disclosure provides a method of producing a scaffold graft material or platelet rich plasma (PRP) graft for tissue repair and/or tissue support or reinforcement in a subject, comprising the steps of:
(i) providing a whole blood sample;
(ii) combining the whole blood sample with an anti-coagulant or surface which initiates coagulation;
(iii) optionally obtaining a platelet rich plasma by subjecting the whole blood sample to a separation force for a first period of time;
(iv) combining the whole blood sample with a coagulation activator;
(v) subjecting the whole blood sample to a separation force while the blood sample is coagulating for a second period of time; and
(vi) separating the pelleted material from the supernatant.

In one example, the method further comprises harvesting the pelleted graft material.

In a first embodiment, the method comprises producing a scaffold graft material, comprising the steps of:
(i) providing a whole blood sample in combination with an anti-coagulant agent and a coagulation activator or surface which initiates coagulation, the arrangement being such that the anti-coagulant agent is sufficient to prevent coagulation of the blood sample within about 5 minutes;
(ii) subjecting the blood sample to a separation force while the blood sample is coagulating to separate the whole blood into a densely coagulated material (pelleted material) and supernatant;
(iii) separating the densely coagulated material from the supernatant to provide the scaffold material for tissue repair and/or tissue support or reinforcement in the subject.

In one example, the method further comprises:
(i) providing a whole blood sample in combination with an anti-coagulant agent and a coagulation activator or surface which initiates coagulation, the arrangement being such that the anti-coagulant agent is sufficient to prevent coagulation of the blood sample within about 5 minutes;
(ii) subjecting the blood sample to a one-step centrifugation at a relative centrifugal force in the range of about 2250×g to about 3750×g while the blood sample is coagulating to form a densely coagulated material and supernatant; and
(iii) separating the densely coagulated material from the supernatant to provide the scaffold material for tissue repair and/or tissue support or reinforcement in the subject.

In one example, the method further comprises (iv) harvesting the densely coagulated material.

In a second embodiment, the method comprises preparation of an autologous PRP graft material, the method comprising the steps of:
(i) collecting a volume of whole blood in a container;
(ii) obtaining a platelet rich plasma (PRP) by subjecting the whole blood sample to a first separation force for a first period of time to separate red blood cells (RBC) from a supernatant comprising PRP;
(iii) combining the whole blood sample with a coagulation activator;
(iv) subjecting the whole blood sample to a second separation force while the blood sample is coagulating for a second period of time to form a graft material.

In one example, the coagulation activator is a gluconate salt. In one example, the gluconate salt is calcium gluconate.

In one example, the first separation force is between 3000-4000 RPM (900-1,600×g) for a first period of time of between 7-12 min.

In one example, the platelet-rich plasma (PRP) and gluconate salt are in a ratio of between 0.5:2-3:8 v/v.

In one example, whole blood is combined with a citrate salt prior to obtaining PRP. In another example, the citrate salt is sodium citrate.

In one example, the PRP concentration is at least 4-6 times the baseline concentration of platelets in the whole blood.

In one example, the second separation force is between 3,500-4,500 RPM (2000-3,200×g) for a second period of time of between 55-75 min.

In one example, the tissue repair and/or support is tissue repair and/or tissue support or reinforcement of a vaginal wall. In one example, the vaginal wall is the anterior vaginal wall. In another example the vaginal wall is the posterior vaginal wall.

The whole blood can comprise a blood sample from a single donor or from multiple donors mixed together to obtain a single blood sample. The blood sample can be obtained from the same subject who will receive the scaffold graft material. Thus, the blood is autologous to the recipient. The blood sample can also be obtained from a non-autologous subject or donor or multiple donors. Thus, the blood sample can be obtained from a heterologous subject or donor or multiple donors. In one example, the blood is collected into a suitable receptacle or container. In one example, the receptacle or container is glass. In another example, the receptacle or container is a borosilicate glass container. In one example the whole blood sample is from the said subject such that the method produces an autologous scaffold graft material for tissue repair and/or support in the subject. The blood sample may therefore be autologous or allogeneic to the subject.

The blood sample may be combined with one or more anti-coagulation agents and one or more coagulants. In a particular example the anti-coagulant and the coagulation activator are combined prior to the addition of whole blood. In one example, the anti-coagulation agent and the coagulation activator and blood sample are provided in sequential order. In one example, the blood sample is combined with the anti-coagulant prior to the addition of coagulant to avoid immediate clotting of the blood.

In one example, the densely coagulated material (i.e. scaffold graft material) according to the first embodiment comprises a substantially homogenous mixture of plasma, platelets, red blood cells and white blood cells. In one example, the densely coagulated material is substantially free of red blood cells and white blood cells. In one example, the proportion of red and white blood cells in the densely coagulated material comprises less than about 10%, preferably less than about 5% red blood cells and less than about 10%, preferably less than about 5% white blood cells. In one example, the densely coagulated material comprises substantially all of the platelets. In one example, the densely coagulated material comprises at least about 90%, at least about 92%, at least about 95%, at least about 96%, at least about 97%, at least about 98%, at least about 99% or 100% of the platelets in the whole blood. In one example, the densely coagulated material comprises about 30% or less of the water content of plasma and plasma proteins and solids. In another example, the densely coagulated material comprises about 95-100% platelets, less than 10% red and white blood cells and about 30% water content of plasma.

In one example, the supernatant according to the first embodiment comprises about 70% plasma. In another example the supernatant comprises at least about 90% of the red blood cells and at least about 90% of the white blood cells in the whole blood.

In one example, the anti-coagulant agent is selected from heparin, ethylenediaminetetraacetic acid (EDTA), citrate, oxalate, thrombin inhibitors or other factor inhibitors. In another example, the anti-coagulation agent is sodium citrate. In one example, the sodium citrate is provided in solution in an amount of 2 ml per 18-20 ml of whole blood. In one example, the sodium citrate is provided as a solution of between about 1% and 5% (v/v), preferably about 3 to 4% (v/v), more preferably about 3.2% (v/v).

In one example, the coagulation activator initiates aggregation of fibrin present in the whole blood. In another example, the coagulation activator is a calcium salt, iron (ferrous) salt, aluminium salt, sodium salt or zinc salt. In another example, the coagulation factor is calcium chloride or calcium sulphate. In another example, the coagulation factor is calcium gluconate. In another example, the calcium gluconate is provided in an amount of 2 mL per 18-20 ml of whole blood. In another example, the calcium gluconate is provided as a solution of about 10%.

In one example according to the first embodiment, the volumes of the whole blood, anti-coagulant agent and coagulation activator in the scaffold graft material are in a ratio of about 8-12:1:1 v/v. In one example, the volumes of the whole blood, anti-coagulant agent and coagulation activator are in a ratio of about 9:1:1 v/v. In one example, the whole blood, sodium citrate, calcium gluconate are in a ratio of about 8-12:1:1 v/v, more preferably 9:1:1 v/v.

In one example according to the first embodiment, the anti-coagulant agent, coagulant activator and whole blood are exposed to a single separation force resulting in the densely coagulated material (i.e. scaffold graft material) and supernatant fractions. In a further example, the method comprises a single centrifugation step.

In one example according to the first embodiment, the densely coagulated material (i.e. scaffold graft material) is an homogenous mixture of platelets, red blood cells, white blood cells and plasma. In another example the densely coagulated material is not a multilayered structure. In another example the densely coagulated material is not a soft jelly-like material.

In one example, the method of the first embodiment does not require immediate clotting of the blood or the step of aggregating the fibrin prior to centrifugation.

The method of the first embodiment may also include modifying the densely coagulated material after harvest. In one example, the modification comprises washing or blotting the material on an absorbent material.

In a second aspect, the disclosure provides a scaffold graft material, produced by or obtainable by, the method of the first aspect. In one example, the scaffold material is an autologous scaffold graft material according to the first embodiment. In one example, the scaffold material is the autologous graft material according to the second embodiment.

In a third aspect, the disclosure provides a scaffold graft material, preferably an autologous scaffold material, for tissue repair and/or tissue support or reinforcement in a subject produced from whole blood, wherein the scaffold material is a flexibly solid non-gel homogenous material comprising about 95-100% platelets, less than 10% red and white blood cells and at least about 30% plasma. The platelets can further include inactivated and activated platelets. In a particular example, the scaffold graft comprises the densely coagulated material as described herein.

In a fourth aspect, the disclosure provides a scaffold graft material for tissue repair and/or tissue support or reinforcement to a pelvic organ in a female subject, wherein the scaffold material is a flexibly solid non-gel material comprising plasma, platelets and fibrin and being substantially free of red and white blood cells, and wherein the scaffold comprises an ultimate tensile strength of at least 3 MPa.

The scaffold graft material can comprise physical properties that allow it to be suturable but also of sufficient strength to provide biomechanical reinforcement to the site of prolapse, for example to the anterior and posterior vaginal walls. In one example, the scaffold material comprises an ultimate tensile strength of between 3 and 5 Mpa. In another example, the scaffold material comprises a suture retention strength of at least about 20N (Newton). In certain examples, the scaffold graft material forms a disc of relatively uniform cross-sectional thickness following centrifugation. In a further example, the diameter of the scaffold disc is in the range of about 3-5 cm, more preferably about 5 cm. In a further example, the thickness of the scaffold disc is about 1.5 to 3 mm). In some examples, the scaffold disc further comprises a surgical glue applied to one or both sides of the disc. In situ this may represent the anterior and/or posterior side. Suitable surgical glues will be familiar to persons skilled in the art. Examples include fibrin glue, albumin-glutaraldehyde or a cyanoacrylate-based tissue adhesive such as octyl-cyanoacrylate.

In a fifth aspect, the disclosure provides an autologous PRP graft material, the graft material comprising platelet rich plasma (PRP) and a gluconate salt, wherein the ratio of the gluconate salt to platelet-rich plasma is between 0.5:2-3:8 v/v. The PRP may be used for skin defects due to trauma, diabetes, or cancer, vascular injury, tendon injury, visceral injury, or any soft tissue damage requiring reinforcement or accelerating tissue reinforcement and augmentation.

In one example according to the fifth aspect, the gluconate salt is calcium gluconate. In one example, the ratio of the gluconate salt to platelet-rich plasma is 1:6 v/v. In another example, the ratio of the gluconate salt to platelet-rich plasma is 1:4 v/v. In a further example, the ratio of the gluconate salt to platelet-rich plasma is 2:6 v/v. In one example, the autologous graft material is a tissue reinforcement autologous graft material. In a further example, the autologous graft material is an autologous graft composite.

In one example, according to the fifth aspect, the autologous graft material (PRP graft) is enriched in platelets. In one example, the PRP graft is substantially free of red blood cells. In one example, the PRP is a jelly-like material.

The scaffold graft material described herein according to the fourth aspect or the autologous PRP graft material according to the fifth aspect, can further comprise optional components that can be added either during or after preparing the scaffold graft material. The scaffold graft material may include one or more materials to assist in strengthening the graft. In one example, the optional component is muscle, for example skeletal muscle. In one example, the graft further comprises a biodegradable scaffold material to further strengthen the graft.

For example, the biodegradable material may be a zinc, copper, magnesium or iron based alloy. In another example, the biodegradable material is a polymer (e.g. biopolymer). The polymer may be synthetic polymer, for example polyorthoester, polyphosphoester, polyanhydride, polyester-amide or polyamide. The polymer may be a natural polymer, for example chitosan, alginate, guar gum, starch, carrageenan, albumin or gelatin. In another example, the optional component is a growth factor. The growth factor may include one or more of vascular endothelial growth factor (VEGF), epidermal growth factor (EGF), insulin-like growth factor (IGF), platelet derived growth factor (PDGF), transforming growth factor beta (TGF-β), and fibroblast growth factor (FGF).

In a sixth aspect, the disclosure provides a method of treating a subject afflicted with pelvic organ prolapse (POP), said method comprising the steps of:
(i) providing a scaffold graft material according to the third, fourth aspect or PRP graft material according to the fifth aspect, or the graft according to the second aspect, and
(ii) affixing the scaffold graft material or the PRP graft material to a site of surgical repair of the prolapse.

In one example, the POP is anterior vaginal wall prolapse. In another example, the POP is uterine prolapse. In another example, the POP is selected from anterior compartment prolapse, posterior compartment prolapse, anterior and posterior prolapse, global prolapse, anterior and apical prolapse, posterior and apical prolapse and apical prolapse.

In some examples, the method of treatment includes applying one or more scaffold graft materials to the site depending on the extent of the injury. The scaffolds can be overlapping on placed side-by-side. In other examples, the method of treatment includes a further treatment subsequent to a failure of previous treatment.

In one example, the subject has one or more of the following age>35 years, body mass index>24, obesity, menopause, previous forcepts delivery, previous caesarean delivery, hysterectomy. In one example, the subject has a concurrent complaint. In another example, the concurrent complaint is selected from voiding dysfunction, recurrent urinary tract infection (UTI), bowel dysfunction, sexual dysfunction, lichen sclerosis, atrophic vaginitis and vulvodynia.

In one example, the method of the sixth aspect further comprises the step of surgical repair of the anterior and/or posterior wall fascia of the vagina. In one example, the surgical repair comprises suturing.

In some examples, one or more scaffold grafts are affixed to the site of surgical repair.

In a seventh aspect, the disclosure provides a method of repairing or augmenting damaged or injured pelvic tissue in a subject, said method comprising the steps of:
(i) providing a scaffold graft material according to the third or fourth aspect or PRP graft material according to the fifth aspect or the graft according to the second aspect; and
(ii) affixing the scaffold graft material or the PRP graft material to a target site in the subject to repair or augment the damaged or injured tissue.

In one example, the target site is a site of injury or defect, more particularly a tear in the vaginal wall epithelium or connective tissue fascia. The tear, injury or defect may occur in the pubocervical fascia (anterior wall fascia), the rectovaginal fascia (posterior wall fascia) or both.

The methods according to the sixth or seventh aspects can include delivering one or more scaffold graft materials or PRP graft materials to the site or surgical repair or target site. Multiple scaffold graft materials or PRP graft materials can be sutured and/or glued together prior to being affixed to the site. In some examples, the scaffold graft material or PRP graft material is shaped into a disc by one or more of stretching, suturing, compressing and/or trimming as required.

In an eighth aspect, the disclosure provides a kit of parts comprising:
(i) a venepuncture needle (e.g., a butterfly needle such as an 18-gauge butterfly needle);
(ii) cannula;
(iii) at least one lidded blood collection container (e.g., specimen container) pre-filled with an anti-coagulant agent and a coagulation activator (or is provided with a surface which initiates coagulation);
(iv) pliers;
(v) at least one syringe pre-filled with a saline solution (e.g., a 20 mL syringe containing sterile 0.9% normal saline for washing); and optionally,
(vi) instructions for preparing the graft according to the first aspect.

In a ninth aspect, the disclosure provides a kit comprising at least one lidded blood collection container pre-filled with an anti-coagulant agent and a coagulation activator, together with instructions for preparation of a scaffold graft material according to the first aspect.

In a tenth aspect, the disclosure provides a kit according to the eighth aspect for use, or when used to treat a subject afflicted with pelvic organ prolapse. In one example the kit further comprises instructions for treating a subject with pelvic organ prolapse.

In some examples, the kit of parts may further comprise a standard sterile dish (e.g., a petri dish) and/or a transfer needle. Further, in some embodiments, the kit of parts may comprise a centrifuge (e.g., a standard benchtop centrifuge).

In an eleventh aspect, the disclosure provides a sterile container when used for the preparation of a scaffold graft material, the sterile container comprising whole blood, an anti-coagulant and a coagulation activator in a ratio of about 8-12:1:1 v/v.

In an twelfth aspect, the disclosure provides a sterile container when used for the preparation of an autologous PRP graft material, the sterile container comprising PRP; a gluconate salt; wherein the ratio of the gluconate salt to platelet-rich plasma is between 0.5:2-3:8 v/v.

### DESCRIPTION OF THE FIGURES

FIG. 1 is a flow chart for producing the scaffold graft material of the disclosure.

FIG. 2 shows a photographic image of a container comprising the scaffold graft material of the disclosure. The pellet comprises the densely coagulated material comprises about 95-100% platelets, at least 10% red and white blood cells and at least about 70% plasma.

FIG. 3 shows a photographic image of a scaffold material produced from whole blood. The scaffold material shown is provided in a substantially disc shape with a diameter (or longest dimension) of about 5 cm, and a substantially uniform thickness of about 2-3 mm. The disc is non-gelatinous in texture, but shows some flexibility and is sufficiently dense so as to provide a robust structure making them broadly applicable to a range of desired sites.

FIG. 4 shows an anatomic figure of the human female pelvis. Locations of the anterior and posterior walls of the vagina are indicated.

FIG. 5 shows a photographic image of the scaffold graft material in situ. (A) scaffold graft sutured to the base of the repaired vaginal connective tissue between the vaginal mucosa and the bladder (B) scaffold graft material glued to the vaginal mucoasa.

### DETAILED DESCRIPTION

### Definitions

The term “and/or”, e.g., “X and/or Y” shall be understood to mean either “X and Y” or “X or Y” and shall be taken to provide explicit support for both means or for either meaning. Furthermore, a list or features including the phrase “and/or” between the second last and last feature means that any one or more of the listed features may be present in any combination.

Reference to the singular forms “a”, “an” and “the” is also understood to imply the inclusion of plural forms unless the context dictates otherwise.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B.

The term “anterior” as mentioned herein refers to the anterior vaginal wall which is located under and adjacent to the bladder.

The term “anterior prolapse” or cystocele refers to a weakness in the connective tissue fascia separating the bladder and vagina and may cause the bladder to bulge into the vagina.

Anterior prolapse is sometimes called prolapsed bladder.

The term “posterior” as mentioned herein refers to the posterior vaginal wall which is located adjacent to the rectum.

The term “posterior prolapse” or rectocele refers to a weakness in the connective tissue fascia separating the rectum and vagina and may cause the rectum to bulge into the vagina.

The term “prolapse” as mentioned herein refers to the process in which a pelvic organ (e.g. uterus) is caused to bulge down into the vagina. It is often also referred to as uterine prolapse and occurs when the pelvic floor muscles and ligaments stretch and weaken and no longer provide enough support for the uterus. As a result the uterus slips down into or protrudes out of the vagina.

The term “subject” as used herein refers to an animal, in one example a mammal, in a further example, a human who will benefit from the scaffold material and methods of the present disclosure. In a particular example, the subject is female.

The term “graft scaffold material” as used herein refers to a densely coagulated homogenous mixture of plasma, platelets and red and white blood cells. The densely coagulated material comprises about 95-100% platelets, at least 10% red and white blood cells and at least about 70% plasma. The graft material is able to be stretched and is suturable or can be glued to the repaired connective tissue of the vaginal wall.

The term “PRP graft” as used herein refers to an enriched platelet composition which is substantially free of red blood cells.

The term “clotting” or “coagulation” as used herein refer to a soft, non-rigid insoluble mass formed when blood gels or the process of forming the soft, nonrigid insoluble blood mass. The term “clot” can apply to the coagulated phase of blood, the soft, coherent, jelly-like mass resulting from the conversion of fibrinogen to fibrin, thereby entrapping blood cells within the coagulated plasma.

The term “aggregated fibrin” as used herein refers to fibrin in its state after clotting or coagulation.

The term “water content of plasma” as used herein refers to the amount of water in the plasma. Plasma comprises approximately 90-92% of water and 8-10% of solids being made up of ions, proteins, dissolved gasses, nutrient molecule and wastes. The proteins include antibody protein, coagulation factors, albumin and fibrinogen.

### Blood Sample

Whole blood is a mixture of liquid and solid components. Blood plasma is the liquid component of blood in which the blood cells (solid components) are suspended. The plasma makes up about 60% of total blood volume and is composed of mostly water (90% by volume) and contains dissolved proteins, glucose, clotting factors, mineral ions, hormones and carbon dioxide. Platelets and blood cells are found in the solid components of whole blood. Platelets and blood proteins work together to stop the bleeding by initiating blood clotting or coagulation and forming a clot over a site of injury. Platelets exert strong procoagulant and antifibrinolytic effects through the release of many growth factors, such as transforming growth factor (TGF-β), platelet derived growth factor (PDGF) and vascular endothelial growth factor (VEGF).

A prevalent blood protein is fibrinogen (Factor 1). As used herein the terms “unaggregated fibrin” and “fibrinogen” are used interchangeably to refer to a precursor to fibrin or in its state prior to clotting or coagulation. In a healthy individual, fibrinogen or unaggregated fibrin has two principle functions, 1) to form bridges for platelets by binding to their surface proteins and 2) a precursor to fibrin. During the clotting process, fibrinogen is converted to fibrin through several steps. First, thrombin cleaves the amino-terminus of the fibrinogen alpha and beta chains to fibrinopeptide A and B, respectively. The resulting fibrin monomers polymerize end to end to form protofibrils, which in turn associate laterally to form fibrin fibers. The fibrin fibers are then capable of associating to form the fibrin gel or clot.

Due to its adhesive properties, a fibrin clot atraumatically connects tissues by forming a strong joint between the tissues and adapts uneven wound surfaces.

Preferably, the whole blood sample is from the subject such that the method produces an autologous scaffold material for tissue repair and/or support in the subject.

### Scaffold Graft Material

The present disclosure is based on the finding that dimensionally stable, homogenous scaffold materials comprising a densely coagulated homogenous mixture of plasma, coagulant, platelets and fibrin which is substantially free of red and white blood cells can be produced which are of sufficient strength to provide mechanical support to site of injury, e.g. site of prolapse.

The scaffold material produced in accordance with the first embodiment of the method of the first aspect is, as has been mentioned above, a flexibly solid non-gel material substantially free of red and white blood cells. The term “substantially” as used herein means less than about 30%, less than about 25%, less than about 20%, less than about 15%, less than about 10%, less than about 5%, less than about 2%. It is preferably further characterised by being sufficiently solid so as to provide biological and mechanical support at the site in which it is to be used (e.g., for a patient with an anterior vaginal wall prolapse, between the fascia and vaginal epithelium), and in some embodiments, the scaffold material is sufficiently solid so as to be suturable; that is, suture thread can be readily passed through the material with a standard suturing needle without any substantial loss of integrity in the material (e.g., the scaffold material remains intact with no tearing, fragmentation or other breakages). However, and as will be described further below, while the scaffold material may be suturable, the scaffold material need not be sutured into the site in which it is to be used.

The subject may be a patient afflicted with a POP such as an anterior vaginal wall prolapse, and in use, the scaffold material may be affixed between the fascia and the vaginal epithelium after a standard surgical repair of the fascial defect at that site has been performed (e.g., by application of sutures to repair the defect). Typically, the whole blood sample provided in step (i) of the method will be collected from the subject shortly before surgery commences (e.g., during the subject's surgical preparation or “pre-op”), so as to preferably enable the scaffold material to be produced during the course of the surgery so that it is available once, or soon after, the surgical repair of the fascial defect has been completed (thereby avoiding need for any storage and/or preservation of the scaffold material). The scaffold material may be affixed to the site of the surgical repair by suitable sutures or, more preferably, the use of an adhesive such as a suitable biocompatible and/or biodegradable adhesive or sealant or a combination thereof (e.g., a bio glue such as fibrin glue, albumin-glutaraldehyde or a cyanoacrylate-based tissue adhesive such as octyl-cyanoacrylate e.g. DERMABOND™) In some examples, the scaffold material may be affixed to the site of surgical repair in a “sandwich” arrangement comprising a first layer of adhesive (applied to the underlying fascia), the scaffold material, and then a second, overlaying, layer of adhesive, before closing the vaginal epithelium (e.g., with suitable sutures) over the scaffold material, so as reduce any risk of seroma. While not wishing to be bound by theory, it is considered that following the surgical procedure, the affixed scaffold material is able to enhance native tissue repair (e.g., by acting as a scaffold to stimulate, for example, stem cell migration and proliferation, and thereby promoting native tissue repair which may involve neovascularisation and collagen formation) and/or provide support (e.g., reinforcement) to the pelvic floor. Accordingly, the scaffold material becomes stronger the longer it remains in the body.

Notwithstanding the above, it is anticipated that the scaffold material produced in accordance with the method of the first aspect, may be widely applied to other circumstances where support and/or enhancement of native tissue repair may be desired. For example, the scaffold material may be produced for treatment of mesh erosion (e.g., in POP patients who have previously undergone surgery involving the use of a synthetic mesh), bowel and bladder injuries, vascular and other non-healing wounds, burns and other skin wounds, diabetic foot, tendon repair, spinal repair, and for soft tissue augmentation.

The scaffold of the first embodiment produced by the method of the first aspect has stable and consistent dimensions. In one example, the scaffold has a diameter of between 3 and 10 cm, preferably between 4 and 8 cm, preferably between 5 and 7 cm, more preferable about 5 cm. In one example, the thickness is about 1.5 to 3 cm.

Depending on the size of the fascia, one or two grafts may be used. In some examples, the grafts are laid side by side. In other example, the grafts are overlapped either wholly or partially if the defect of the fascia is more substantial.

The scaffold material can also be enlarged by joining, layering or bonding multiple constructs together using standard techniques such as suturing, heating, stapling, and gluing with biological glue, or a combination of these methods.

In some examples, a surgical glue is applied to one or both faces of the scaffold graft material. In some examples, one or more procoagulant or crosslinking factors may be added to the scaffold or the glue. In some examples, the procoagulant may be selected from calcium ions or salts, Factor I, Factor II, Factor III, Factor IV, Factor V, Factor VII, Factor X, Factor XI, Factor XII, Factor XIII, thrombokinase, proconvertin, antihemophilic globulin, prothrombase, collage, arachidonic acid and fibrinase. In some examples, the cross-linking agent is selected from a condensing agent, an aldehyde (e.g. glutaraldehyde) and carbodiimide EDC (1-ethyl-3(3 dimethyl aminopropyl). In further examples, one or more growth factors or agents that facilitate stem cell migration to the scaffold may be added to the scaffold or glue. These may include any of the factors elaborated from the platelets as described earlier (e.g. VEGF, FGF, EGF, BMP, PDGF etc.).

### Production of the Scaffold Graft Material

The whole blood sample described herein is preferably provided in combination with an anti-coagulant agent and a coagulation activator or surface which initiates coagulation. By “in combination with” it is meant that the anti-coagulation agent and the coagulation activator are provided separately to the whole blood or sequentially to the whole blood. Conveniently, this may be achieved by collecting the whole blood sample from the patient (using a standard blood collection methodology) into a suitable blood collection container pre-filled with an anticoagulant agent and a coagulation activator, or into a suitable blood collection container pre-filled with an anticoagulant agent and provided with a surface which initiates coagulation. The volume of the whole blood sample may be in the range of about 10 mL to about 50 mL in size, more preferably about 15 ml to 25 mL, in order to provide a scaffold material of a useful size. For the production of a scaffold material suitable for the treatment of POP, a whole blood sample of about 20 mL is particularly suitable (e.g., an 18 mL, 19 mL, 20 mL, 21 mL or 22 mL sample).

With a whole blood sample of about 20 mL and the use of a cylindrical blood collection container with a diameter of 5-7 cm during the centrifugation step, the present method may enable the production of, for example, a scaffold material of circular or ovoid shape with a diameter (or longest dimension) of about 5 cm. Such discs of the scaffold material will typically be about 1.5 to 3 mm, preferably about 2-3 mm in thickness. However, those skilled in the art will readily appreciate that the scaffold material may be provided in other shapes and/or dimensions, and that this may be readily directed by the shape of the base of the chosen container used for the centrifugation step. Thus, the scaffold material may, in some embodiments, be square- or rectangular-shaped of, for example, 3 cm×3 cm, 4 cm×4 cm, 5 cm×5 cm, 5 cm×3 cm or 7 cm×5 cm etc. The thickness of the scaffold material will typically be in the range of about 1.5 mm to about 4 mm (preferably, 2 mm to 3 mm) to ensure flexibility and, preferably, sufficient solidity so as to be suturable.

Suitable surfaces capable of initiating coagulation (also referred herein as a coagulation-initiating surface) are well known to those skilled in the art and include, for example, certain negatively-charged surfaces, and including glass (such as may be provided by providing a blood collection container with, for example, a glass bead or an internal glass surface of a wall of the container), which may activate factor XII to initiate the intrinsic pathway of blood coagulation. Other suitable coagulation-initiating surfaces may include surfaces comprising kaolin and various forms of silica (Margolis J, J Physiol 137:95-109, 1957), and surfaces comprising various organic polymers such as one selected from polypropylene, polyethylene, polycarbonate, polyamide, polyurethane and styrene (see, for example, US Patent Application Publication No. 2021/0077533).

After obtaining the blood, the blood can be placed in a container. Typically, the blood may be collected in a suitable blood collection tube which is known in the art. Exemplary examples include various types of glass, such as borosilicate glass. Borosilicate glass is a type of glass having silica and boron oxide as the main glass-forming constituents.

However, preferably, the methods described herein comprise the use of one or more coagulation activators (e.g., by conveniently collecting the whole blood sample into a suitable blood collection container pre-filled with a coagulation activator). Suitable coagulation activators (also known as haemostatic agents) are well known to those skilled in the art and include, for example, various calcium salts (Mellanby FRS and CLG Pratt, Proc R Soc Lond Series B 128(851):201-213, 1940) such as calcium chloride and calcium sulphate. One preferred coagulation activator of this kind for use in the present method is calcium gluconate. Other suitable coagulation factors include various iron, aluminium, sodium and zinc salts including, for example, ferric or ferrous sulphate, aluminium sulphate, alum (ie KAI(SO₄)₂, aluminium chloride, sodium gluconate and zinc chloride). Those skilled in the art will be able to readily identify a suitable amount of a coagulation activator(s) for use in the combination of step (i). The amount may, of course, vary depending upon the particular coagulation activator(s), but may be for example, where the coagulation activator is a calcium salt, an amount providing about 15 μmoles to about 50 μmoles calcium ions to the whole blood, more preferably, about 20 μmoles to about 25 μmoles. In some embodiments of the present method, where the coagulation activator is calcium gluconate (10%), the calcium gluconate may be provided in an amount of about 2 ml for an 18 mL to 20 mL amount of whole blood.

The anti-coagulant agent of the methods described herein may be selected from any of the suitable anti-coagulant agents (or combinations thereof) known to those skilled in the art. Some suitable examples include heparin and ethylenediaminetetraacetic acid (EDTA) (e.g., potassium EDTA) which have been widely used for whole blood sample collection. Another widely used anti-coagulant agent, preferred for use in the present methods, is sodium citrate. In some embodiments, the sodium citrate may be provided as a solution with a concentration in the range of about 1% to about 5% v/v, more preferably in the range of 3% to 4% v/v (e.g., 3.2% v/v sodium citrate or 3.8% v/v sodium citrate).

The anti-coagulant agent(s) (such as a sodium citrate solution) is provided in an amount sufficient to prevent coagulation of the blood sample within about 5 minutes. That is, the relative amount of the anti-coagulant agent(s) that is provided is sufficient to counteract the effect of the coagulation activator or coagulation-initiating surface and the natural propensity of whole blood to coagulate outside of the body, for a short term (e.g., up to about 5 minutes) so as to readily permit the blood sample (in combination with the anti-coagulant agent(s) and coagulation activator/coagulation-initiating surface) to be transferred to a centrifuge for the centrifugation step of the present method, without any substantial coagulation occurring, since the method requires that the blood sample is coagulating during the centrifugation to form a densely coagulated material and supernatant. It is well within the routine skill of those skilled in the art to identify a suitable relative amount of the anti-coagulant agent(s) by, for example, routine titration of the amount of the anti-coagulant agent required(s) (i.e., to prevent coagulation of the blood sample within about 5 minutes) when provided with a certain known amount of a coagulant activator (or in the presence of a coagulation-initiating surface) in a suitable volume of whole blood (e.g., 18 mL or 20 mL etc.). For example, it has been found by the present inventors that for a combination comprising 18 mL of whole blood and 2 mL of 10% calcium gluconate solution, an amount of 2 mL of 3.2% (v/v) sodium citrate (as the anticoagulant agent) prevents any substantial coagulation of the blood sample within about 5 minutes.

Thus, in some embodiments, the method of the first aspect comprises providing a whole blood sample in combination with an anti-coagulant agent and coagulation activator, wherein the volumes of the whole blood, anti-coagulant agent and coagulation activator are in a ratio of about 8-12:1:1 v/v. Further, in some more specific embodiments, the method of the first aspect comprises providing a whole blood sample in combination with sodium citrate (3.2% solution) and calcium gluconate (10% solution), wherein the volumes of the whole blood, anti-coagulant agent and coagulation activator are in a ratio of about 8-12:1:1 v/v, more preferably about 9:1:1 v/v.

In some examples, the whole blood sample is firstly mixed with the anti-coagulant agent to avoid immediate clotting of the blood and then the blood and anti-coagulant agent are mixed with the coagulation activator. In some examples, the blood sample is exposed to the anti-coagulant during or after collection of the sample to permit ease of handling the blood.

In one example the whole blood, coagulation activator and anti-coagulant are mixed in a centrifuge container or bottle having a diameter of 5 cm. Such bottles are known in the art. In one example, the bottle is a polypropylene bottle. In other examples, the bottle is a polycarbonate or polysulfone bottle.

The methods described herein comprise subjecting the blood sample, anti-coagulant and coagulation activator (in a suitable container) to a one-step centrifugation. The separation force preferably separates the blood into a densely coagulated material (located at, or pelleted to the bottom of the centrifugation container or bottle) and a supernatant. In one example, the blood sample, anti-coagulant and coagulation activator are centrifuged at a relative centrifugal force in the range of about 2250G to about 3750G, more preferably in the range of 2500G to 3500G, while the blood sample is coagulating.

Centrifugation may be applied for a determinate period of time. In one example, the determinate period of time is sufficient time to allow for the separation of the whole blood into the densely coagulated material and supernatant. Preferably, this centrifugation, which may conveniently be conducted on a standard benchtop centrifuge with the blood sample contained within a suitable blood collection container, is conducted for a period in the range of about 30 to about 90 minutes, more preferably in the range of 45 to 60 minutes, more preferably about 46 minutes, 47 minutes, 48 minutes, 49 minutes, 50 minutes, 52 minutes, 55 minutes, or 60 minutes. At the end of the centrifugation, the whole blood sample is present as a densely coagulated material (comprising plasma, platelets and fibrin) and a supernatant (comprising red and white blood cells, and a small amount of plasma). The preferred duration of the centrifugation step (i.e. in the range of about 30 to about 90 minutes), is to enable the scaffold material to be produced, in most cases, during the course of the surgery.

In another example, the sample is centrifuged at between 4,500 to 4,800 RPM on a bench centrifuge with radius of about 15 cm.

Since the method comprises a one-step centrifugation, the method offers a relatively simplified method of production. Accordingly, the method is suitable for automation. For example, blood can be collected using a butterfly needle attached to a vacutainer or collected directly into a centrifugation container (under sterile conditions), the container pre-filled with the coagulation activator and anti-coagulant. In one example, there is no other separate centrifugation step involved in the method, and in particular, there is no centrifugation step (or any other step) to first produce a platelet-rich plasma (PRP), which means that in the method of the first aspect, the centrifugation is conducted on a sample comprising a normal physiological amount of platelets present in the subject's whole blood.

The methods described herein also comprise separating or harvesting the densely coagulated material from the supernatant to provide the scaffold material for tissue repair and/or support in the subject. This may be achieved by any routine procedure such as, for example, carefully removing and transferring the densely coagulated material from the container in which the centrifugation step was conducted (e.g., by using sterile forceps/pliers) to a standard sterile dish (e.g., a petri dish) where it may, optionally, be washed (e.g., with a sterile saline solution) before use. The construct may be optionally modified for use e.g. by trimming, blotting, suturing, stretching or compression.

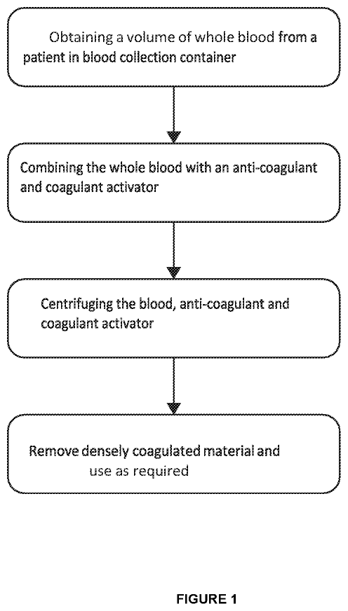

FIG. 1 provides a schematic of the production process.

In some examples, the method may further comprise a step of attaching the scaffold material to a suitable sheet material which may provide, for example, mechanical strength or reinforcement. As such, the sheet material may, for example, form a “backing layer” to the scaffold material. Suitable sheet materials include any of those that will be apparent to those skilled in the art and may, for example, comprise a medical-grade foam backing sheet or other biocompatible and/or biodegradable sheet material including those comprising, for example, synthetic polymers such as poly(glycolic acid), poly(lactic acid) and polyethylene glycol (PEG) or naturally-occurring polymers such as alginate, chitosan, starch, dextran and albumen. The scaffold material may be attached to the suitable sheet material by bonding, laminating or any of the other means or techniques well known to those skilled in the art (including, for example, bonding with an adhesive (e.g., spot bonding), preferably a biocompatible adhesive or sealant such as fibrin glue, and chemical treatment to achieve cross-linking between elements of the scaffold material and sheet material). In some examples, the scaffold material may be attached to muscle prior to use in the subject.

### Measuring Scaffold Strength

The suture retention strength refers to the force required to pull a suture out of the scaffold graft material described herein. It relates to the maximum force required (N) to pull the suture from the graft which can be measured using a technical tensile tester. In one example, the scaffold graft has a suture retention strength of at least 20N (Newtons) at least 30N, at least 40N, or more preferably at least 50N, as measured using the suture retention strength test described by Adelman D M et al., Plast Reconstr Surg Glob Open 2(5):e155, 2014).

In some examples, the scaffold graft material has a uniaxial tensile strength of at least 3 Mpa, at least 4 Mpa or at least 5 Mpa or greater. The uniaxial tensile strength is the stress applied to a sample until failure (e.g. splitting, separating or cracking). The force can be applied as either a tension or compression. There are a number of devices available for measuring the tensile strength which can range from simple mechanical devices to automated computerised machines. Examples of such devices are described, in for example Gunter S et al., (2021) Eng. Res. Express. 3:045055.

The basic premise is to place a sample of material between two fixtures called “grips” which clamp the material. A weight is then applied to the material gripped at one end while the other end is fixed. One example includes the Instron ElectroPuls E100.

### Optional Components

The scaffold material may comprise optional components that can be added during or after preparing the material. For example, the scaffold material may be treated with additives or drugs prior to implantation, for example to promote the formation of new tissue after implantation. Thus, for example, stem cells, growth factors, cytokines, extracellular matrix components, and other bioactive materials can be added to the substrate to promote healing and formation of new tissue. In one example, VEGF is employed to promote the formation of new vascular tissue. Growth factors and other additives (e.g., epidermal growth factor (EGF), heparin-binding epidermal-like growth factor (HBGF), fibroblast growth factor (FGF), stromal cell derived factor, insulin-like growth factor (IGF), transforming growth factor (TGF-β1), platelet-derived growth factor (PDGF-ap), macrophage inflammatory proteins 1 alpha (MIP-1 alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-alpha, and TNF-beta, leptin, leukemia inhibitory factor (LIF), endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, cytokines, genes, proteins, and the like) can be added.

Other useful additives include antibacterial agents such as antibiotics.

### Delivery

Methods of delivering the scaffold material to a target site in the subject can include, but are not limited to, placement of the fibrin construct within or on a target site. The scaffold material can be held at the target site by methods such as, but not limited to, implantation, suturing and/or gluing the scaffold to the target site.

The scaffold material can be delivered to the target site and kept in place at the target site by methods used in the art. Such methods can include, but are not limited to, suturing techniques using absorbable synthetic suture material such as the biocompatible polymer is polyglactin and polyglycolic acid, manufactured as Vicryl™ by Ethicon Co., Somerville, N.J. (See e.g., Craig P. H., Williams J. A., Davis K. W., et al.: A Biological Comparison of Polyglactin 910 and Polyglycolic Acid Synthetic Absorbable Sutures. Surg. 141; 1010, (1975)), staples, joining with biological glues and/or tissue adhesives such as synthetic adhesives, glues based on cyanoacrylates (2-butyl cyanoacrylate, 2-octyl cyanoacrylate), or on synthetic polymers, and others contain biological materials such as collagen or fibrin (See e.g., U.S. Pat. Nos. 5,844,016, 5,874,500; 5,744,545; 5,550,187 and 6,730,299). A person skilled in the art will appreciate that various combinations of such techniques can be used as well.

### Kits

In a fifth aspect, the present disclosure provides a kit of parts comprising: a venepuncture needle (e.g., a butterfly needle such as an 18-gauge butterfly needle); cannula; at least one lidded blood collection container (e.g., specimen container) pre-filled with an anti-coagulant agent and a coagulation activator (or is provided with surface which initiates coagulation); pliers; at least one syringe pre-filled with a saline solution for washing (e.g., a 20 mL syringe containing sterile 0.9% normal saline); and optionally, instructions for use of the kit in the method of the first aspect.

In some embodiments, the kit of parts may further comprise a standard sterile dish (e.g., a petri dish) and/or a transfer needle (otherwise known as a hypodermic needle that may be used for collecting or transferring blood). Further, in some embodiments, the kit of parts may comprise a centrifuge (e.g., a standard benchtop centrifuge).

The kit of parts may be packaged in any of the convenient sterile forms known to those skilled in the art including blister packs (e.g., where multiple cavities or pockets (“blisters”) are provided to accommodate one or more of the components of the kit). Where the kit of parts comprises a centrifuge, then typically, the centrifuge will be packaged separately (again in a sterile form) from the other kit components. The kit of parts, as packaged in such manners, will be suitable for use, for example, within an operating theatre (provided that the kit comprises a centrifuge, or the operating theatre is otherwise provided with a suitable centrifuge), and as such, may enable the production of a scaffold material according to the present disclosure to be produced in the operating theatre in which it is to be used.

The scaffold materials, methods and kit of the present disclosure are hereinafter further described by way of the following non-limiting examples.

### EXAMPLES

### Example 1 Preparation of Densely Coagulated Scaffold Material for Tissue Repair or Tissue Augmentation

### Materials and Methods

### Preparation of Sodium Citrate Solution

The sodium citrate solution was prepared by adding 25.703 g of Sodium Citrate dehydrate to 800 ml of distilled water. After mixing, 2.421 g of citric acid was added. After combining, the pH was then adjusted to physiological pH using hydrogen chloride (HCl) or sodium hydroxide (NaOH). Distilled water was then added to a final volume of 1 L to produce a 3.2% (v/v) sodium citrate solution.

### Preparation of Calcium Gluconate Solution

The calcium gluconate solution (10% w/v) was obtained from commercial sources. Each ml contains 0.095 g calcium gluconate as monohydrate, equivalent to 0.212 mmol calcium. The product also contains an amount of the excipient calcium saccharate equivalent to 0.0112 mmol calcium per mL (or 0.112 mmol calcium per 10 mL). Total calcium content: 0.223 mmol per mL (2.23 mmol per 10 mL).

### Preparation of Densely Coagulated Scaffold Graft Material

Using a standard blood collection methodology (e.g., venepuncture), 18 mL of whole blood was collected from a patient into a sterile medical-grade lidded container with a diameter of 5-7 cm (e.g., 70 mL sterile specimen containers with a 5.5 cm diameter such as a Sarstedt sterile 70 mL yellow cap specimen jar, 44 mm×55 mm (Sarstedt Australia Pty Ltd, Mawson Lakes, SA, Australia)) pre-filled with 2 mL buffered sodium citrate solution (3.2% v/v) and 2 mL calcium gluconate (10%; a solution containing 95.3 mg/mL of calcium gluconate gluconate, and 3 mg/mL of calcium saccharate; wherein each one mL contains 8.9 mg of calcium equivalent to 0.22 mmoles or 0.44 mEq of calcium ions). The container is then placed in a suitable centrifuge such as a standard benchtop centrifuge (e.g., LuXiangyi TDZ5-WS Tabletop Low Speed Centrifuge; Shanghai Lu Xiangyi Centrifuge Instrument Co., Ltd, Shanghai, China) and subjected to a single step centrifugation at 2500-3500×g at room temperature for 50 to 60 minutes (e.g., about 4000-4,800 rpm for 1 hour in the abovementioned LuXiangyi Tabletop Centrifuge). Ideally, the centrifugation step is commenced within about 5 to 15 minutes of collection of the whole blood samples.

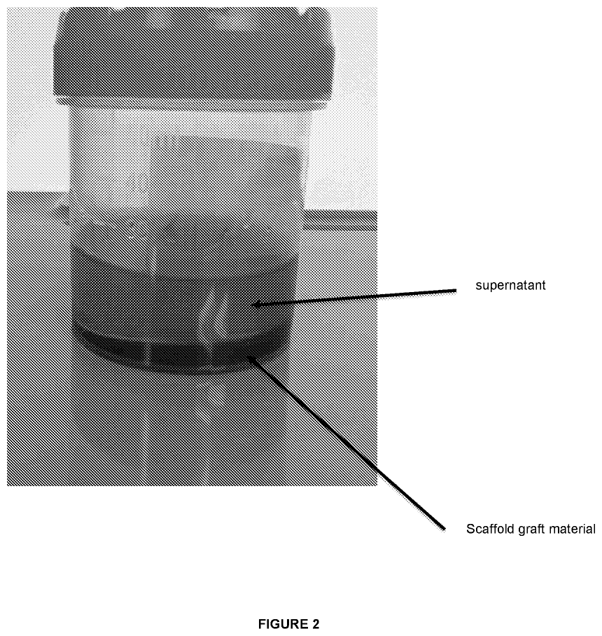

During the centrifugation, the whole blood samples begin to coagulate and thicken to form the scaffold material while the fluid (mainly water) and red and white blood cells (and a small amount of plasma) form the supernatant (see FIG. 2). At the end of the centrifugation step, the lid of the container is removed (in a sterile manner) and the disc of dense scaffold material at the base of the container carefully removed from the container with forceps/pliers (leaving behind the supernatant comprising mostly red and white blood cells, and a small amount of plasma) and transferred to a standard sterile dish (e.g., a petri dish). Optionally, the scaffold can be washed with 50 mL of sterile 0.9% normal saline (to remove residual supernatant and blood cells). The disc of dense scaffold material comprising most of plasma, substantially all of the platelets (and blood proteins) and less than about 10% red and white blood cells is then ready for use and may be anchored (affixed) at a site where native tissue repair and/or support is desired by, for example, biocompatible and/or biodegradable sutures and/or adhesive (e.g., fibrin glue).

### Results and Discussion

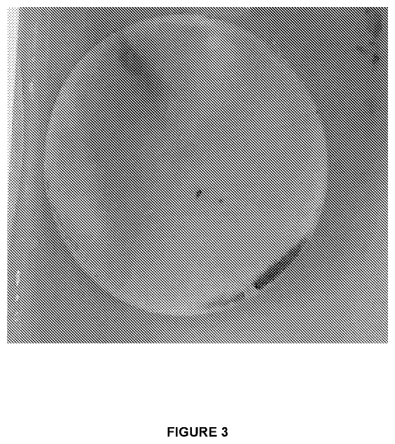

An image of a disc of dense scaffold material produced in accordance with the method described in the preceding paragraph is shown in FIG. 3. The disc is of a substantially circular shape with a diameter of about 5 cm. The disc is about 2-3 mm in thickness and is substantially of even thickness throughout the disc (i.e. a little thinner on the periphery). The disc of scaffold material is non-gelatinous in texture, but shows some flexibility making them broadly applicable to a range of desired sites. The scaffold material is substantially free of red and white blood cells, but a small number of red blood cells may sometimes be on the surface of the scaffold material and these can be readily removed by washing with normal saline. Standard braided suture thread (e.g., suture thread composed of a copolymer of glycolide (90%) and L-lactide (10%) such as Vicryl™ sutures (Ethicon, Raritan, NJ, United States of America) and barbed suture thread composed of glycolide, dioxanone and trimethyl carbonate such as V-Loc™ suture thread (Covidien, Dublin, Ireland) can be readily passed through the discs with a standard circle suturing needle, without substantial loss of integrity. That is, the disc scaffold material remains intact and may provide support at the site of anchoring.

### Example 2 Treatment of Patient with Anterior Vaginal Wall Prolapse

A 53 year old female patient with a severe visible prolapse of the anterior vaginal wall was admitted to the hospital and prepared for surgery. This patient would have previously been a candidate for vaginal mesh repair. The patient completed an Australian Pelvic Floor Questionnaire (APFQ) prior to surgery.

During the preparation for surgery, two 9 mL whole blood samples were initially collected from the patient via venepuncture into two 10 mL blood tubes and then transferred into a lidded specimen container with a 5.5 cm diameter (so that the container contained a total of 18 mL of whole blood) and immediately used to prepare the autologous scaffold material (by a method substantially as described in Example 1) in a designated room attached to the operating theatre in a sterile fashion. The turnaround time from the taking of the whole blood samples to the provision of the scaffold material to the surgeon in the operating theatre was about 50-70 minutes.

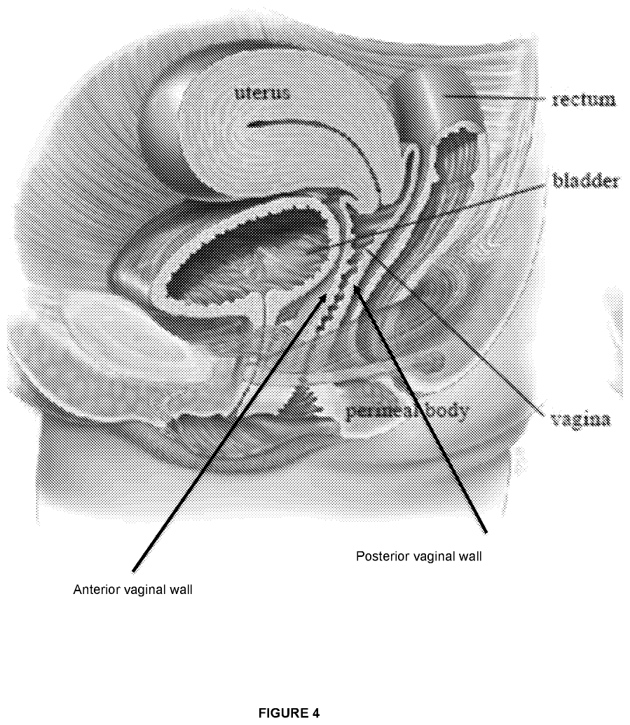

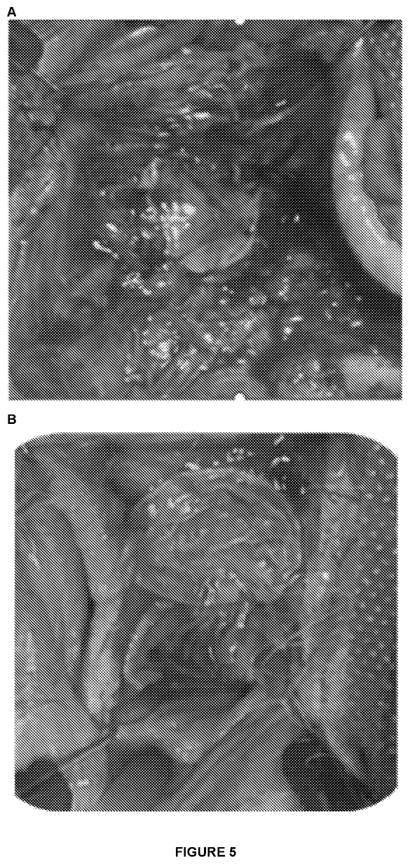

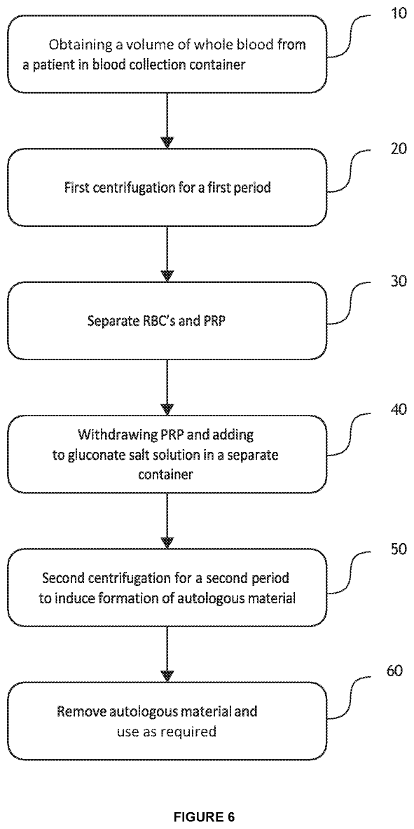

Following general anaesthetic, surgery on the patient was commenced by making a midline incision into the vaginal epithelium (mucosa) of the anterior vaginal wall (see location in FIG. 4) followed by repair of the underlying fascial defect by use of V-Loc™ 2-0 sutures (Covidien). Prior to closing the epithelium, the scaffold material was anchored to the site by first applying a suitable quantity of fibrin glue (e.g., Tisseel; Baxter International, Deerfield, IL, United States of America) to the site on the underlying fascia, introducing the scaffold material to the fibrin glue, and then applying a second suitable quantity of fibrin glue over the top of the scaffold material. The vaginal epithelium was then closed with suitable sutures (e.g., 2-0 Vicryl™ sutures (polyglactin 910, Ethicon)). This is shown in FIG. 5). At completion of the surgery, a betadine-soaked or PRP-soaked vaginal pack and in-dwelling catheter were placed in the patient overnight prior to a formal trial of void to assess the ability of the patient's bladder to fully empty. The patient was then discharged on a five-day course of oral antibiotic agents (e.g., amoxicillin and clavulanic acid). Six weeks later, a post-operative review of symptoms and physical examination was performed to check on surgical success and vaginal integrity.

The patient was then followed up for post-surgical review at 6 weeks, between 3-6 months and at 12 months.

### Example 3 Calcium Analysis of Supernatant Associated with the Scaffold Graft Material

The supernatant obtained following production of the scaffold graft according to Example 1 was analysed for calcium concentration on a Siemens Advia/Atellic chemistry system. The supernatant was found to contain >8.0 mmol/L calcium.

This indicated that there was a very high concentration of calcium in the supernatant rather than in the scaffold suggesting that toxicity of the scaffold when implanted into the subject will be very low.

### Example 4 Preparation of Platelet-Rich-Plasma (PRP) Autologous Graft

The following example describes the preparation and use of an autologous graft prepared from blood in the augmentation of NTR at the time of POP surgery aimed as a biological support to regenerate damaged native tissue.

### Patient Characteristics

The details of the patients are set out in Table 1 below.

TABLE 1
Patient characteristics

| | | |
|---|---|---|
| Age | 62.6 | (±12.4, 34-84) |
| BMI | 29.9 | (±5.1, 18-40) |
| Obesity | 36 | (55.4%) |
| Presenting complaint | | |
| Anterior Compartment Prolapse | 3 | (3.1%) |
| Posterior Compartment Prolapse | 9 | (9.2%) |
| Anterior and Posterior Prolapse | 30 | (30.6%) |
| Global Prolapse | 33 | (33.7%) |
| Anterior and Apical | 8 | (8.2%) |
| Posterior and Apical | 7 | (7.1%) |
| Apical Prolapse | 1 | (1.0%) |
| Concurrent complaint | | |
| Voiding dysfunction | 90 | (91.8%) |
| Recurrent UTI | 6 | (5.71%) |
| Prolapse symptoms | 87 | (92.6%) |
| Bowel dysfunction | 91 | (96.8) |
| Sexual symptoms | 42 | (40.0%) |
| Lichen sclerosis | 7 | (7.1%) |
| Atrophic vaginitis | 81 | (82.7%) |
| Valvodynia | 1 | (1.0%) |
| Parity | 2.3 | (±0.9, 90, 91.8%) |
| Gravidity | 2.1 | (±1.4, 66, 67.3%) |
| Patients with previous vaginal deliveries | 89 | (90.8%) |
| Number of vaginal births per patient | 2.2 | (±1.0) |
| Previous forceps delivery | 5 | (5.1%) |
| Previous caesarean delivery | 12 | (12.2%) |
| Post-menopausal | 75 | (76.5%) |
| Previous pelvic floor surgery (total) | 65 | (66.3%) |
| Previous pelvic floor | 51 | (52.0%) |
| surgery (involving hysterectomy) | | |
| Previous pelvic floor | 14 | (14.3%) |
| surgery (excluding hysterectomy) | | |
| Previous hysterectomy | 37 | (37.8%) |
| Previous mesh surgery | 10 | (10.2%) |
| Previous PRP treatment | 42 | (42.9%) |
| Previous laser treatment | 60 | (61.2%) |
| Previous urinary incontinence surgery | 11 | (11.2%) |
| Previous stress urinary incontinence surgery | 5 | (5.1%) |
| Previous urge urinary incontinence surgery | 3 | (3.1%) |
| Previous mixed urinary incontinence surgery | 3 | (3.1%) |
| Sexually Active | 39 | (46%) |

BMI, Body Mass Index.
Data shown as n (valid percent accounting for missing values)
Age and BMI mean (standard deviation, range) where age is in years, and BMI is kg/m2
Parity, Gravidity shown as mean (standard deviation, n, % of cases applicable)
Number of vaginal births per patient, mean (standard deviation)
Previous hysterectomy - not necessarily with previous pelvic floor surgery

### Materials

PRP tubes were obtained from Surecell (https://www.surecell.com.au/surecell-prp-tubes). 8 ml with sodium citrate. Calcium gluconate (2.2 millimoles of calcium ions in 10 mL) purchased from Whelping Supplies, two sterile medical-grade plastic collecting containers with diameter of 5.5 cm.

The centrifuge used is a TDZ5-WS Tabletop low speed centrifuge.

### Methods

A single centre prospective cohort study of women who underwent native tissue repair (NTR) for any POP between 2018 and 2021 was undertaken. This study was approved by Bellberry Limited. All women signed a written, informed consent form. Patients were recommended topical oestrogen or PRP as a conservative management for vaginal atrophy Patients who were unable to take topical oestrogen or participate in the pre-treatment of the vaginal epithelium with PRP were excluded from the trial. Comprehensive assessments and surgical procedures were performed by two surgeons. Comprehensive assessments and surgical procedures were performed by two surgeons (FBW and TTN).

Eligibility for enrolment in the cohort initially included patients with recurrent POP or patients with severe primary prolapse who were candidates for mesh repair (either vaginal or laparoscopic abdominal mesh). Patients were initially referred for consideration of vaginal mesh however, during the waiting period, the vaginal mesh products were withdrawn from the Australian market and many patients opted against abdominal mesh consequently.

All patients were pre-treated with a standard regime of 3 courses of PRP and twice weekly application of topical Ostridiol cream (Ovestin 1 mg/g, Organon Ltd) for 3 months.

By reference to FIG. 6, at the commencement of the case, patient blood was drawn into collection containers (10) which were then centrifuged for a first period (20) (3500 RPM for 9 minutes) to separate the RBC from PRP (30) supernatant. The PRP supernatant was withdrawn and added to calcium gluconate in a pre-defined ratio in a sterile container (40) prior to further centrifugation for a second period (50) (4000 RPM for 45 minutes) during which the material begins to coagulate and thicken to form a solid suturable graft that is malleable and able to be handled without disruption to structure of the graft material. The resulting autologous material formed is then removed (60) and can be used in the appropriate procedure.

Following general anaesthetic, a midline incision into the vaginal epithelium is made and the underlying fascial defect is repaired using V-Lok 2-0 suture (Medtronic). Prior to closing the epithelium, the autologous membrane material is sutured to the underlying fascia. The vaginal epithelium is then closed with 2-0 Vicryl (polyglactin 910, Ethicon). Where required an apical suspension was also performed with or without concurrent hysterectomy. Apical suspension was achieved through either a sacrospinous fixation placed with a capio slim absorbable monofilament suture (Boston Scientific), a laparoscopic uterosacral ligament fixation (either hysteropexy or colpopexy with 2.0 V-Loc delayed absorbable suture) or a laparoscopic sacrocolpopexy using Restorelle mesh (Coloplast Pty Ltd). A hysterectomy was performed following a discussion with the patient or for any suspected pathology.

At completion of surgery a vaginal pack and in-dwelling catheter were placed overnight prior to a formal trial of void. Routine strict postoperative instructions were provided to the patient including advice on physical activity, bowel habit and intercourse. All patients were discharged on five days of Amoxicillin and Clavulanic acid.

Patients follow up was conducted at 6 weeks, 3-6 months, and 12 months. They were advised to monitor and immediately report any adverse events. All adverse events regardless of their relationship with surgery were recorded.

Prospective data was collected on an initial enrolment sheet and entered the research database. All patients completed the Australian Pelvic Floor Questionnaire (APFQ) prior to surgery and at each follow up visit. The APFQ is a validated tool which integrates, bladder, bowel, and sexual functioning, pelvic organ prolapse severity, bothersomeness and condition-specific quality of life (Kapoor D S, Thakar R, Sultan A H, Oliver R. Conservative versus surgical management of prolapse: what dictates patient choice? Int Urogynecol J Pelvic Floor Dysfunct. 2009; 20(10):1157-61). All examination findings were recorded using the Pelvic Organ Prolapse Quantification System (POP-Q) (Mowat A E, Maher C. Transvaginal mesh: let's not repeat the mistakes of the past. Aust N Z J Obstet Gynaecol. 2017; 57(1):108-10). Patient demographics including age, body mass index (BMI), menopausal status, parity and previous surgeries were also recorded.

### Blood Preparation Protocol

1. 20 ml of whole blood was withdrawn from the patient into proprietary collection tubes containing sodium citrate (e.g. BD Vacutainer tubes) to assist in separating plasma and the platelets from the red blood cells and the white blood cells, creating a PRP at least 4-6 times the baseline concentration of platelets.
2. The two tubes containing whole blood are placed into a centrifuge in a way that is opposite each other for balance.
3. The tubes are centrifuged for a period of 9 minutes (pre-set button) at 4,000 RPM to separate the plasma from the white and red blood cells.
4. The tubes are gently removed without being shaken to avoid mixing of the platelets and RBCs. A sterile 10 mls syringe and 27-gauge needle are used to aspirate the plasma without contamination of the red cells and white cells. This is done for both tubes.
5. A sterile medical-grade plastic container with a diameter of 5.5 cm is used to mix plasma obtained from the two tubes, which is approximately about 8 mls of PRP and 2 mls of calcium gluconate %. The lid of the plastic container is closed for insertion into a different centrifuge.
6. The tube is centrifuged for a second period at a 45-degree angle at 4,000 RPM for 45 minutes.
7. After 45 minutes, the container is removed. The lid is opened in a sterile manner/environment. The product tis then transported into a sterile dish to be used in a sterile manner.

### Statistical Analysis

Statistical analysis was performed using Stata version 16.1 (StataCorp, Texas, USA). Means and standard deviations (SD's) were calculated for continuous data and proportions for categorical data. The normality assumption was visually checked by frequency histogram and normal Q-Q plot for continuous measurements. The Anderson-Darling test was also performed to test the normality assumption. For non-normally distributed data, median, interquartile ranges and Wilcoxon signed-rank models will also be reported.

A multivariate mixed effect model was applied to examine the primary and secondary outcomes. As the outcome occurs for each individual with repeated time points, the mixed effect models will capture both fixed effects and random effects within the hierarchical structure of the data. Thus, models were accounted for the clustering in patients using mixed (for interval scale data—domain score) and melogit (for binary outcomes—QOL). Patients were treated as random effects, and main effects were group, time and group x time interaction. Models were adjusted by age and BMI as they are clinically important. The two-sided test was performed for all analyses and the level of significance was set at p<0.05.

### Results

Between 2018 and 2021, 105 patients underwent surgery for pelvic organ prolapse, but only 97 were included for analyses with a minimum 6 week follow up (T2). The characteristics of the population and the surgeries performed are characterised in Table 1. The mean age was 62.6 (34-84), and the mean body mass index was 29.9 kg/m2 (18-40 kg/m2) with half the cohort classified with obesity (55.4%). 66.3% of patients had previous pelvic organ prolapse repair surgery with most having an associated hysterectomy (52.0%). Most patients had global prolapse (32.4%).

The surgery characteristics performed on this cohort are outlined in Table 2 below. 89 (91.8%) patients undertook concurrent apical prolapse repair. Of these, 31 underwent a laparoscopic uterosacral fixation while, 6 underwent sacrospinous fixation and 6 underwent a laparoscopic mesh sacrocolpopexy.

TABLE 2
Surgery Characteristics

| Surgery | No. of patients | |
|---|---|---|
| Anterior Only | 6 | (6.2%) |
| Posterior Only | 27 | (27.8%) |
| Anterior and Posterior | 64 | (66.0%) |
| Concurrent Apical Repair | 94 | (96.9%) |
| Concurrent Hysterectomy | 37 | (38.1%) |
| Concurrent SUI Repair | 11 | (11.3%) |
| Sling Procedure | 3 | (3.1%) |
| Botox Injection | 5 | (5.2%) |
| Periurethral Injection (Bulking Agent) | 2 | (2.1%) |
| Kelly's Sutures | 1 | (1.0%) |

Scores in all domains improved significantly following surgery. There was also a significant improvement in the degree of bother (QOL) scored across the domains of bladder, bowel and pelvic organ prolapse as demonstrated in Table 3.

TABLE 3
Mean AFPQ domain and bother scores at baseline (T1), 6 weeks
(T2), 3-6 months (T3), and 12 months post-treatment (T4).

| | Baseline | 6 weeks | 3-6 months | 12 months |
|---|---|---|---|---|
| Domain | (T1) | (T2) | (T3) | (T4) |
| Bladder | 12.85 ± 8.10 | 6.38 ± 6.18 | 8.60 ± 6.78 | 5.52 6 ± .37 |
| Bowel | 10.09 ± 6.74 | 7.09 ± 4.82 | 7.09 ± 6.06 | 5.87 ± 5.44 |
| Prolapse | 7.28 ± 4.31 | 0.88 ± 1.93 | 1.18 ± 2.25 | 0.88 ± 1.65 |
| Sexual | 3.35 ± 4.24 | 1.08 ± 2.32 | 0.49 ± 1.49 | 0.37 ± 1.31 |

Values are expressed as mean (standard deviation)

The reduction in domain scores over the follow-up period demonstrated an overarching trend with significant reduction post-operation at 6 weeks (T2) remaining static until 6-12 months (T4) suggesting effective improvement lasting until 6-12 months. At 6 weeks (T2), the average reduction was as follows: bladder (−6.62, P<0.001, 95% CI), bowel (−3.10, P=0.001, 95% CI), prolapse (−6.38, P<0.001, 95% CI), and sexual (−2.23, P<0.001, 95% CI). For total domain outcomes, only up to 51-63 (52.6%-65.6%) were eligible for 6 week, 48-85 (49.5-87.6%) for 3-6 months, and 33-51 (34.0%-52.6%) for 12 months follow-up analyses. Due to 66.0% of patients undertaking combined anterior and posterior repairs, the effects of individual compartment repairs on the improvement in the domain scores was unable to be examined.

Regarding bladder bother, binary logit regression demonstrated that the likelihood of complete resolution to score 0 post-operatively at T2 was 85% (OR 0.15, P<0.001, 95% CI [0.07, 0.32]). The mean predicted probability of reporting some degree of bother was 0.83 at baseline and decreased by about half (0.43) at T2 remaining significantly static throughout follow up. This is reflected by how the mean change in bladder bother score at T2 was −0.95 points with a vast increase in the proportion of patients experiencing no bother at all from 16.7% at T1 to 57.4% at T2. This proportion remained relatively stable until T4. There is a general decrease in the proportion of patients with score 2 and 3 of bother. However, the proportion is relatively unchanged for those with score 1 (34.4% at T1 and 31.5% at T2) suggesting there is notable improvement in bladder bother particularly for those with severe bother at baseline but post-operation they are more likely to have mild bother (score 1) if no bother (score 0). The improvement after T2 remained relatively stable through to T4 except for a mild increase in proportions of score 2 and 3 at T3 which correlated with the bladder domain scores which suggests that a booster may be required for some patients around 3-6 months.

For bowel bother, binary logit regression showed that the likelihood of complete resolution to score 0 post-operatively at T2 was 68% (OR 0.32, P<0.001, 95% CI [0.16,0.64]) with lasting significant improvement over follow up. The mean predicted probability of reporting some degree of bother was 0.67 at baseline and decreased to 0.39 at T2 remaining static throughout although slightly increasing over later follow ups. By T2, the mean change in bowel bother score was −0.71 points with the proportion of patients with no bother (score 1) at T1 improving from 33.3% to 61.1% post-operation (T2) and remaining stable until T4 (57.1%) However, after the initial improvement post-operation, bowel bother proportions gradually rise from 3.75 to 10.7% by T4 indicative of fading treatment effect.

Binary logit regression for prolapse demonstrated that the likelihood of complete resolution to score 0 post-operatively at T2 was 97% (OR 0.03, P<0.001, 95% CI [0.01,0.08]) with significantly lasting effect throughout follow up. The mean predicted probability of reporting some degree of bother was 0.87 at baseline and decreased to 0.18 at T2 remaining significantly static over follow up. Post-operatively (T2), the mean change in prolapse bother score improved by −1.86 points from baseline. The proportion of patients with no bother improved from 12.8% at baseline to 82.4% at T2 remaining stable throughout follow-up. About half (51.2%) reported severe bother at baseline but the proportion dropped to 2.0% at T2 but rose to 9.1% at T3 then dropped to 0.0% at T4. This suggests vast subjective improvement post-operatively but some may need booster treatment at T3.

Sexual bother was only applicable to 58 (59.8%) of patients. Binary logit regression showed that the likelihood of complete resolution to score 0 post-operatively at T2 was 61% (OR 0.39, P=0.05, 95% CI [0.15,1.00]) with significantly static improvement throughout follow-up. The mean predicted probability of reporting some degree of bother is 0.63 at baseline and decreases to 0.41 at T2 remaining static throughout. At T2, the mean change in sexual bother score was −0.68. The proportion of patients with no bother improved from 36.2% at baseline to 59.3% at T2 and continued to improve to 88.2% at T4. For those who followed-up at T3 and T4, there were no patients with moderate or greatly bothered (score 3 or 4) sexual QOL.

The summary of frequencies for bother scores are shows in Table 4.

TABLE 4
Frequencies for bother scores at baseline (T1), 6 weeks
(T2), 3-6 months (T3), and 12 months post-treatment (T4).

| Bother Score | T1 | | T2 | | T3 | | T4 | |
|---|---|---|---|---|---|---|---|---|
| Bladder | | | | | | | | |
| 0 | 15 | (16.7%) | 31 | (57.4%) | 21 | (45.7%) | 16 | (57.1%) |
| 1 | 31 | (34.4%) | 17 | (31.5%) | 14 | (30.4%) | 10 | (35.7%) |
| 2 | 27 | (30.0%) | 5 | (9.3%) | 8 | (17.4%) | 1 | (3.6%) |
| 3 | 17 | (18.9%) | 1 | (1.9%) | 3 | (6.5%) | 1 | (3.6%) |
| Mean | 1.51 ± 0.99 | | 0.56 ± 0.74 | | 0.85 ± 0.94 | | 0.54 ± 0.74 | |
| Median | 1.00 | | 0.00 | | 1.00 | | 0.00 | |
| Range | 0-3 | | 0-3 | | 0-3 | | 0-3 | |
| N | 90 | | 54 | | 46 | | 28 | |
| Mean | −0.95 | | | | | | | |
| change T1-T2 | | | | | | | | |
| Bowel | | | | | | | | |
| 0 | 30 | (33.3%) | 33 | (61.1%) | 25 | (56.8%) | 16 | (57.1%) |
| 1 | 22 | (24.4%) | 13 | (24.1%) | 14 | (31.8%) | 8 | (28.6%) |
| 2 | 21 | (23.3%) | 6 | (11.1%) | 2 | (4.5%) | 1 | (3.6%) |
| 3 | 17 | (18.9%) | 2 | (3.7%) | 3 | (6.8%) | 3 | (10.7%) |
| Mean | 1.28 ± 1.12 | | 0.57 ± 0.84 | | 0.61 ± 0.87 | | 0.68 ± 0.98 | |
| Median | 1.00 | | 0.00 | | 0.00 | | 0.00 | |
| Range | 0-3 | | 0-3 | | 0-3 | | 0-3 | |
| N | 90 | | 54 | | 44 | | 28 | |
| Mean | −0.71 | | | | | | | |
| change T1-T2 | | | | | | | | |
| Prolapse | | | | | | | | |
| 0 | 11 | (12.8%) | 42 | (82.4%) | 34 | (77.3%) | 23 | (76.7%) |
| 1 | 11 | (12.8%) | 5 | (9.8%) | 6 | (13.6%) | 3 | (10.0%) |
| 2 | 20 | (23.3%) | 3 | (5.9%) | 0 | (0.0%) | 4 | (13.3%) |
| 3 | 44 | (51.2%) | 1 | (2.0%) | 4 | (9.1%) | 0 | (0.0%) |
| Mean | 2.13 ± 1.07 | | 0.27 ± 0.67 | | 0.41 ± 0.90 | | 0.37 ± 0.72 | |
| Median | 3.00 | | 0.00 | | 0.00 | | 0.00 | |
| Range | 0-3 | | 0-3 | | 0-3 | | 0-2 | |
| N | 86 | | 51 | | 44 | | 30 | |
| Mean | −1.86 | | | | | | | |
| change T1-T2 | | | | | | | | |
| Sexual | | | | | | | | |
| 0 | 21 | (36.2%) | 16 | (59.3%) | 15 | (68.2%) | 15 | (88.2%) |
| 1 | 8 | (13.8%) | 5 | (18.5%) | 4 | (18.2%) | 1 | (5.9%) |
| 2 | 10 | (17.2%) | 2 | (7.4%) | 3 | (13.6%) | 1 | (5.9%) |
| 3 | 18 | (31.0%) | 3 | (11.1%) | 0 | (0.0%) | 0 | (0.0%) |
| 4 | 1 | (1.7%) | 1 | (13.7%) | 0 | (0.0%) | 0 | (0.0%) |

| N | 58 | 54 | 46 | 28 |
|---|---|---|---|---|
| Mean | 1.49 ± 1.31 | 0.81 ± 1.21 | 0.45 ± 0.74 | 0.37 ± 0.72 |
| Range | 0-4 | 0-4 | 0-2 | 0-2 |

| | |
|---|---|
| Mean | −0.68 |
| change T1-T2 | |

Values are expressed as frequency (%), mean (standard deviation), median (interquartile range), range.

Follow up for bladder bother scores at T1 was 90 (92.8%) but limited over the course of months as T2 was 54 (55.75), T3 was 46 (47.4%), and T4 was 28 (28.9%). These values were similar across the other bother domains and worse for sexual bother domains likely due to patient privacy. Therefore, the QOL data interpretation is more reliable for earlier follow-up times.

Non-parametric testing was utilised with descriptive statistics for POPQ stages due to skewed pre- and post-operation populations. The mean POPQ stage at presentation was 2.76±0.575 (median 3, range 1-4), post-operatively the mean was 0.19±0.583 (median 0, range 0-3) with a statistically significant decrease in mean 2.57 (r=0.89, P<0.001) with strong effect. Models demonstrated that 97.9% of the cohort had improvement (decrease) in scores. This high value is attributable to how any decrease in score is regarded as improvement. The remaining 2.1% found no change in their POPQ stage. By their latest follow-up, 89.7% were POPQ 0, 3.1% were POPQ 1, 6.2% were POPQ 2, and 1.0% were POPQ 4 according to descriptive statistics.

The mean atrophic vaginitis (AV) severity score at baseline was 2.54±0.724 (median 3, range 0-3), post-operatively the mean was 0.68±0.680 (median 1, range 0-3), with a statistically significant decrease in mean 1.86 (r=0.86, p<0.001) with strong effect. 93.9% of the cohort demonstrated improvement. Follow up rate was 82.7%. Binary logit regression showed that the chance of complete resolution of AV severity was 94% post-operatively (OR 0.06, P<0.001, 95% CI [0.02,0.17]). The majority (65.6%) of patients scored 3 points whereas post-operation the majority of patients scored 1 (48.3%) closely followed by no atrophic vaginitis (score 0, 42.5%).

Although no significant interaction, post-menopausal women had higher bladder, bowel, and prolapse domain scores than pre-menopausal women over follow-up. However, for bladder domain there was a significant difference in the mean change (3.89 points higher for post-menopausal group, P=0.009, 95% CI) between pre- and post-menopausal women at 6-12 months (T4). For prolapse domain, post-menopausal women had higher scores at baseline (2.19 mean difference, P=0.020, 95% CI) and borderline significant difference at 3-6 months (T2) (0.72 mean difference, P=0.006, 95% CI) indicating significantly higher severity of prolapse symptoms at baseline with similar improvement to the pre-menopausal women except at the 3-6 month mark. This also suggests the treatment is equally as effective for post-menopausal women in the longer term. For sexual domain, the post menopausal group had significantly lower baseline scores (−2.54, P=0.035, 95% CI) suggesting that pre-menopausal women report more sexual domain symptoms. There was no significant interaction between menopausal status, all bother scores, and AV severity. Overall, this indicates that the effects of intervention are significantly less effective than for post-menopausal women at 6-12 months (bladder) and 3-6 months (prolapse) suggesting they may need a booster treatment sooner than pre-menopausal women.

Previous surgery was divided into the group with hysterectomy and without hysterectomy. The hysterectomy group had significantly higher bladder domain scores at each time point except T3 but by T4 their scores are 4.79 points lower on average (P=0.025, 95% CI) compared to patients without previous surgery or who had surgery without previous hysterectomy indicative of a vast improvement in the 6-12 month time frame. The non-hysterectomy group also had overall higher bladder domain scores compared to the hysterectomy group although non-significant except at T4 where the non-hysterectomy group scored −4.78 points lower (P=0.003, 95% CI) mirroring the trend of the hysterectomy group.

For bowel domain, the non-hysterectomy group had lower scores overall but only significant at baseline (−2.98, P=0.050, 95% CI [−6.00,0.5]) and T4 (−4.67, P=0.007, 95% CI [−8.05,−1.28]). Scores were generally higher for the hysterectomy group but not significantly.

For prolapse domain, the non-hysterectomy group had non significantly overall higher scores except at T3 where the average difference is significantly lower than that of the non-surgical group and hysterectomy group suggesting that treatment is maximally beneficial for this group at 3-6 months (mean difference −1.16, P<0.001, 95% CI [−1.82,−0.51]). Although non-significant, the hysterectomy group also had slightly higher overall scores until T4 were the mean difference dips below the non-surgical group and hysterectomy group (−0.29, P=0.617, 95% CI [−1.42,0.84]).

For sexual domain, the non hysterectomy group had generally lower scores but significantly at 3-6 months suggesting best results then (mean difference −5.80, P=0.003, 95% CI) There was non significant effect for hysterectomy group although they started at a lower baseline and had higher or equivocal scores throughout follow-up suggesting less effective change in their sexual domain.

Secondary analysis demonstrated that age and BMI had no effect on domain scores, bother scores, and AV severity. Binary logit models showed that previous surgery with or without hysterectomy had no significant effect on all bother scores and AV severity. POPQ stage was not applicable for logit regression modelling hence secondary analyses was not excluded.

Major complications were reported for 8.2% of patients and half the cohort experienced common minor complications post operatively (49.0%). The most frequent complication was urinary tract infection in 9% of patients.

This study defined failure of the graft as a recurrence of prolapse in the same compartment the autologous graft was applied to. A recurrence without failure was defined as de novo prolapse in another compartment unrelated to the graft. Major complications occurred in eight patients, all requiring reoperation although six cases were related to recurrences and three of them related to failure of the graft. There were three recurrences, all anterior, managed conservatively. A total of nine recurrences (9.3%) were recorded. Regarding failure of graft, there were five cases (4.1%) all of which were anterior with one concurrent apical. There were four recurrence without failure cases all being apical except one anterior. A total of 6 reoperations were required for recurrences with or without failure.

As is now clear, the autologous graft material of the present invention is of great benefit as it provides a graft material based on a patient's own blood, which can be readily produced without the need for additional components such as thrombin or hyaluronic acid, or complicated time-consuming process and greatly increases the rate of soft tissue reinforcement and augmentation in acting as a scaffold to attract stem cell migration, proliferation, therefore promoting neovascularisation and collagen formation, which significantly benefits patients. Further, the graft material was found to be safe and efficacious.

The autologous and biodegradable properties of the autologous graft material of the present invention prevent the inducing foreign body reactions compared to synthetic mesh, accelerate treatments, reduce morbidity, and enhance functional recovery in several medical fields including, but not limited to, skin defects, burns, vascular, musculoskeletal, visceral, spinal surgery and gynaecology. The method of the present invention may also be automated due to the lack of complicated processes and materials. In this manner a patient's blood could be inserted into the automated machine, either directly via syringe or via suitable container and the automated machine would then carry out the required steps including centrifugation according to predefined parameters to produce the autologous graft material. This would mean that prior to the commencement of patient treatment/surgery the required volume of patient blood would be withdrawn and the autologous graft material can be produced in advance or close to the time it was required for use without the need for substantial manual intervention.

It will be appreciated by those skilled in the art that the scaffold material, methods, and kit of the present disclosure are not restricted in their use to the particular application described. Neither are the scaffold material, methods and kit of the disclosure restricted in any preferred embodiment with regard to the particular elements and/or features described or depicted herein. It will be further appreciated that the scaffold material, methods, and kit are not limited to the embodiment or embodiments disclosed, but are capable of numerous rearrangements, modifications and substitutions without departing from the scope of the present disclosure as set forth and defined by the following claims.

### Example 5 Histological Analysis of Vaginal Epithelium at Site of the Scaffold Graft

A sample of vaginal epithelium was obtained from a female subject aged 71 who had received a scaffold graft prepared according to Example 1. A section measuring 15×7×3 mm with a central grey plaque measuring 6×3 mm was submitted for histology. The section showed a piece of fibromuscular tissue covered with cytologically orderly stratified squamous epithelium lacking a granular cell layer. There was no demonstration of significant inflammation, polarisable extraneous material or fungal elements and hence no significant abnormality of the vaginal epithelium.

A sample of vaginal mucosa was obtained from a female subject aged 80 years who had received a scaffold graft prepared according to Example 1. A section measuring 30×20 mm and up to 2 mm thickness was submitted for histology. The section showed epidermis and dermis. The epidermls showed mild acanthosis, hyperkeratosis and focal parakeratosis. No squamous dysplasmia was seen and no viral cytopathic changes were identified. There was no basal cell degeneration or vacuolation and the underlying stroma was unremarkable. No sclerosis or inflammation was seen. There was no evidence of lichen sclerosus, vaginal intraepithelial neoplasia or invasive carcinoma.

A sample of vaginal mucosa was obtained from a female subject aged 84 who had received a PRP graft prepared according to Example 4. A section measuring 62 mm in greatest length and 32 mm in greatest width with thickness of 3 mm was submitted for histology. The section showed squamous lined mucosa with submucosal stroma and some smooth muscle at the deep aspect. The squamous cells showed an orderly maturation sequence with no dysplasia, mitotic activity or koilocytic change. No atrophic features were seen. The subepithelial stroma was normal and there were no features of lichen sclerosus. Some of the vessels near the stromal/muscle interface show hyalinisation (glassy appearance) with almost complete obliteration of some vessel lumina. Some of these hyalinised areas include tiny calcium deposits, occasional foam cells and a rare giant cell. The vessel walls were intact and there was no vasculitis.

In summary, no abnormal histology was seen following surgery.

1-2. (canceled)

3. A method for producing a scaffold graft material for tissue repair and/or tissue support or reinforcement in a subject, comprising the steps of:
(i) providing a whole blood sample in combination with an anti-coagulant agent and a coagulation activator or surface which initiates coagulation, the arrangement being such that the anti-coagulant agent is sufficient to prevent coagulation of the blood sample within about 5 minutes;
(ii) subjecting the blood sample to a separation force while the blood sample is coagulating to separate the whole blood into a densely coagulated material (pelleted material) and supernatant;
(iii) separating the densely coagulated material from the supernatant to provide the scaffold graft material for tissue repair and/or tissue support or reinforcement in the subject.

4. The method according to claim 3, wherein the blood sample is subjected to a one-step centrifugation in the range of about 2250×g to 3750×g.

5. The method according to claim 4, wherein the centrifugation period is between 50 and 60 mins.

6. The method according to claim 1, further comprising (iv) harvesting the densely coagulated material.

7-9. (canceled)

10. The method according to claim 1 wherein tissue repair and/or support is tissue repair and/or tissue support or reinforcement of a vaginal wall.

11. The method according to claim 1, wherein the blood is autologous to the subject.

12. The method according to claim 1, wherein the scaffold material comprises a substantially homogenous mixture of plasma, platelets, red blood cells and white blood cells.

13. The method according to claim 12, wherein
(i) the densely coagulated material comprises at least about 95% of the platelets in the whole blood; and/or
(ii) the densely coagulated material comprises about 30% or less of the water content of plasma; and/or
(iii) the densely coagulated material comprises less than 10% red and white blood cells.

14-15. (canceled)

16. The method according to claim 1, wherein the coagulation activator is selected from a calcium salt, iron (ferrous) salt, aluminium salt, sodium salt or zinc salt and the anti-coagulant agent is selected from heparin, ethylenediaminetetraacetic acid (EDTA), citrate, oxalate, and thrombin inhibitor.

17. The method according to claim 16, wherein the coagulation activator is calcium gluconate and the anti-coagulant is sodium citrate.

18. The method of claim 1, wherein the volumes of the whole blood, anti-coagulant agent and coagulation activator are in a ratio of about 8-12:1:1 v/v.

19. The method of claim 18, wherein the volumes of the whole blood, sodium citrate and calcium gluconate are in a ratio of about 9:1:1 v/v.

20. A scaffold graft material, produced by, or obtainable by, the method of claim 1.

21. A scaffold graft material for tissue repair and/or tissue support or reinforcement in a subject produced from whole blood, wherein the scaffold material is a flexibly solid non-gel homogenous material comprising about 95-100% platelets, less than 10% red and white blood cells and at least about 30% water content of plasma.

22. The scaffold graft material according to claim 21 which comprises an ultimate tensile strength of at least 3 Mpa.

23. The scaffold graft material according to claim 21, which comprises a suture retention strength of at least 20N.

24. The scaffold graft material according to claim 21, wherein the graft forms a disc of relatively uniform cross-sectional thickness following centrifugation and having a diameter in the range of about 3-5 cm, and thickness of between 1.5 and 3 mm.

25. (canceled)

26. The scaffold graft according to claim 24, further comprising a surgical glue applied to one or both sides of the disc.

27-28. (canceled)

29. A method of treating a subject afflicted with pelvic organ prolapse (POP), said method comprising the steps of:
(i) providing a scaffold graft material according to claim 21; and
(ii) affixing the scaffold graft material to a site of surgical repair of the prolapse.

30-31. (canceled)

32. A method of repairing or augmenting damaged or injured pelvic tissue in a subject, said method comprising the steps of:
(i) providing a scaffold graft material according to claim 21; and
(ii) affixing the scaffold graft material to a target site in the subject to repair or augment the damaged or injured tissue.

33-39. (canceled)
